(19) United States
(12) Patent Application Publication
(10) Pub. No.: US 2018/0325688 A1
(43) Pub. Date: Nov. 15, 2018

(54) SYSTEMS AND METHODS FOR ENDOSCOPIC VERTEBRAL FUSION

(71) Applicants: Beth Israel Deaconess Medical Center, Inc., Boston, MA (US); Massachusetts Institute of Technology, Cambridge, MA (US)

(72) Inventors: Kevin J. McGuire, Needham, MA (US); Nadia Gen San Cheng, San Mateo, CA (US); Maria Telleria, Lake Orion, MI (US)

(21) Appl. No.: 16/023,588

(22) Filed: Jun. 29, 2018

Related U.S. Application Data
(63) Continuation of application No. 14/355,503, filed on Apr. 30, 2014, now abandoned.
(60) Provisional application No. 61/565,153, filed on Nov. 30, 2011.

(51) Int. Cl. A61F 2/44; A61B 17/16; A61B 17/295; A61B 17/32; A61F 2/46; A61B 17/70; A61B 17/88; A61B 18/14; A61B 17/3207; A61B 17/00; A61B 90/00; A61B 18/00; A61F 2/30; A61B 90/30; A61F 2/48

(52) U.S. Cl. CPC A61F 2/441; A61F 2002/4495; A61B 17/16; A61B 17/1604; A61B 17/1671; A61B 17/295; A61B 17/32002; A61F 2/4611; A61B 17/7097; A61B 17/8805; A61B 17/8811; A61B 17/8841; A61B 17/8855; A61B 18/1482; A61B 17/320708; A61B 2017/00261; A61B 2017/00199; A61B 90/361; A61B 2017/003; A61B 2017/320004; A61B 2018/00339; A61B 2218/002; A61B 2218/007; A61F 2002/30548; A61F 2002/30581; A61F 2002/30583; A61F 2002/30607; A61F 2002/30616; A61F 2002/444; A61F 2002/4475; A61B 2090/306; A61F 2002/485; A61F 2/442

(57) ABSTRACT

The present invention relates to systems and methods for treatment of the spine. Preferred embodiments utilize a tool system to remove tissue from the intervertebral space, a system for delivery of inflatable membranes or balloons into the intervertebral space and a fluid management system to provide for controlled delivery of fluids into the balloons. A visualization system can also be used to observe tissue removal and balloon placement.

### CROSS REFERENCE TO RELATED APPLICATIONS

This application is a continuation of U.S. application Ser. No. 14/355,503, filed on Apr. 30, 2014, which is a 35 U.S.C. § 371 national stage filing of International Application No. PCT/US2012/067399, filed on Nov. 30, 2012, which claims priority to U.S. Provisional Application 61/565,153 filed on Nov. 30, 2011, the entire contents of all of the above applications being incorporated herein by reference.

### STATEMENT REGARDING FEDERALLY SPONSORED RESEARCH OR DEVELOPMENT

This invention was made with Government support under Contract No. W81XWH-07-2-0011 awarded by the U.S. Army Medical Research and Material Command. The Government has certain rights in the invention.

### BACKGROUND OF THE INVENTION

Various methods and tools have been developed for the treatment of the spinal column, including those for the removal and replacement of the intervertebral disc between adjacent vertebrae of the spine. The necessity for this type of surgical treatment can arise due to disease, degeneration and/or injury that can significantly impair the routine functioning of a patient's spinal column and can generate debilitating pain. Existing techniques can involve bone grafting techniques and discectomy. Options for replacement of the disc include artificial disc implantation or the introduction of a cage into the intervertebral space. However, the insertion of a cage is highly invasive and the cage structure has provided limited mobility.

Existing techniques utilize a large open surgical incision to access the annular region. Some have proposed the use of a balloon that is inserted into the vertebral body for inflation in an attempt to stabilize the spinal column, reduce pain and enhance post-operative mobility. Thus far such methods have failed to gain acceptance. Improvements are needed to provide a safe and effective treatment that better emulates normal functioning of the spine.

### SUMMARY OF THE INVENTION

The present invention relates generally to systems and methods for treatment of the spine. A preferred embodiment includes a system providing endoscopic access to the intervertebral space, tools for the removal of tissue from the intervertebral space, a system for the endoscopic delivery of a plurality of inflatable or expandable members into the intervertebral space and a fluid delivery system to control inflation of the inflatable members using a plurality of fluids.

A preferred embodiment employs a tool kit having a plurality of tools for removing inner portions of the annulus fibrosus and the nucleus pulposus from between a pair of vertebral bodies to provide a cavity of sufficient size. It is preferred to remove a region of cartilaginous endplate material to punctuate bleeding. It is necessary to remove at least 60%, and preferably over 80% of the disc material while leaving the outer annulus intact, so that the implanted material can stabilize the spacing between vertebrae and reduce the risk of further injury.

Endoscopic access is generally accomplished using a cannula providing a portal that is less than 8 mm in diameter. Thus, the elements of the system being introduced into the intervertebral space must be inserted through the cannula or an endoscope working channel. Additionally, a second endoscopic access region can also be used to provide visualization of the intervertebral space as well as a second port to introduce tools or prosthetic devices. Due to the small diameter of the access port(s) and the relatively large volume of tissue to be removed, an important aspect of preferred embodiments of the invention involves the use of an articulating end for the tools being introduced into the space. Due to the varying characteristics of the material being removed, different tool elements must be inserted through the narrow channel and yet be moved over an arcuate path within the intervertebral space to access the material to be removed. In certain cases, a sufficient mechanical force must be imparted using the tool to dislodge fragments of tissue. A combination of rongeurs, curettes, rakes and ring curettes with distal ends having an angular adjustment range can be used for tissue removal. An adjustable angle shaver can also be used to shave or condition the vertebral endplates around the corners. The shaver can be a mechanical shaver with a rotating or oscillating blade or cutting edge, or alternatively, can comprise an RF electrode assembly. Light from a laser coupled into the annular cavity can also be delivered using a fiber optic probe to assist in the removal of tissue or conditioning of the cavity. The combination of mechanical, electro-mechanical and optical instruments provide for complete discectomy through at least one cannula. The system for tissue removal can also include the delivery and removal of fluid to assist in the removal of tissue debris that has been detached from the internal surfaces of the cavity.

Following removal of tools from the cavity, the device for implantation of the prosthesis can then be inserted into the cannula. A preferred embodiment utilizes a probe having a first balloon and a second balloon mounted at the distal end. The first balloon is preferably shaped to conform to the patients' intervertebral cavity when expanded into position. As it is desirable to verify proper spacing of the vertebrae, a measurement of the precise spacing, such as between adjacent lumbar vertebrae, can be performed using one balloon that has been inflated with removable fluid. The first balloon is mounted onto the distal end of the probe which has one or more internal lumens to provide for fluid delivery and removal. The second balloon can also be mounted on the probe shaft proximal to the connector that releasably connects the first balloon to the probe. Thus, both balloons are inserted using a single probe. The second balloon is used to provide proper spacing between the endplates of adjacent vertebral bodies while the first balloon is positioned and filled with cement. The second balloon is subsequently removed after the first balloon is filled with an implant material such as bone cement and has hardened into a fixed geometry. A fluid control system can be used to control delivery of fluids such as saline and bone cement. After the probe is detached from the first balloon, additional bone cement or bone graft is inserted to fill to cavity and seal the annulus. The balloons used for a particular patient can have a size and shape selected to conform to the particular intervertebral space which can be determined using medical imaging procedures, such as computed tomography x-ray scanning systems. Fluoroscopy can be used during and after balloon insertion to verify proper positioning of the implant. The balloon or inserted fluid can serve as a fluoroscopic marker.

### BRIEF DESCRIPTION OF THE DRAWINGS

FIG. 1 schematically illustrates a system for treatment of the spine in accordance with the invention;

FIG. 2 illustrates a procedure for treatment of the spine in accordance with the invention;

FIG. 3A-3B illustrate tool elements for the removal of tissue from an intervertebral space;

FIGS. 4A-4E illustrate a tool with a rotatable articulating tip for the removal of tissue;

FIGS. 5A-5B illustrate a tool using an articulating tip with a rotating shaft in accordance with a preferred embodiment of the invention;

FIGS. 6A-6E illustrate a balloon delivery system in accordance with the invention;

FIG. 7 illustrates a perspective view of the balloon system;

FIG. 8 illustrates a connector for the fluid channel system in accordance with the invention;

FIG. 9 illustrates an example of a mechanical fluid delivery system using a plurality of syringes;

FIG. 10 is a preferred embodiment of a fluid delivery system that measures the amount of fluid and the delivery pressure.

### DETAILED DESCRIPTION OF THE INVENTION

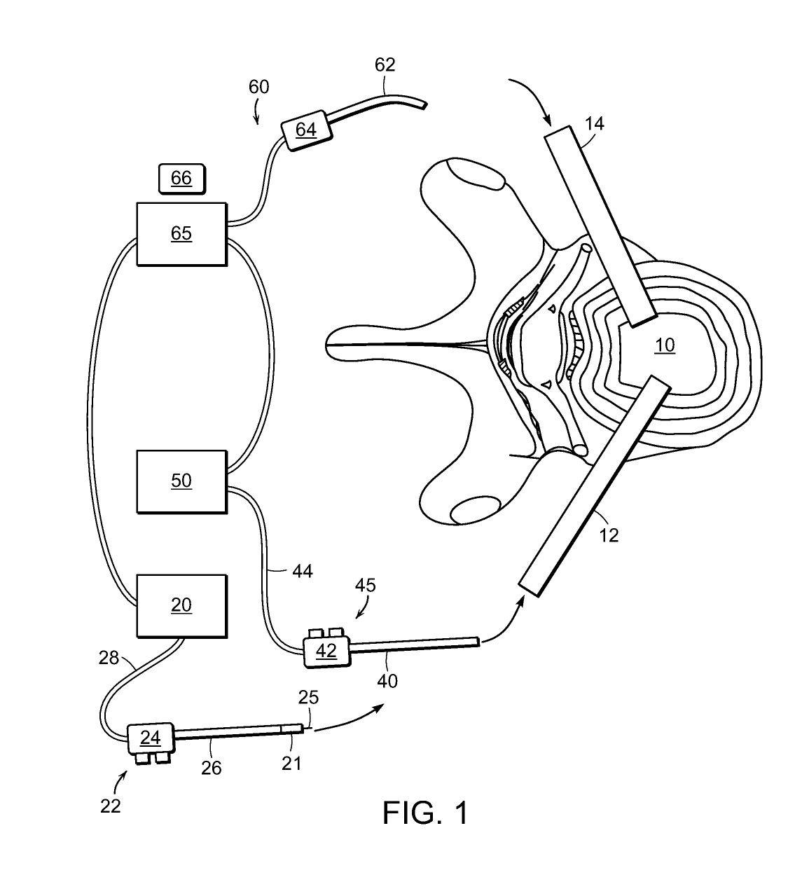

The present invention provides systems and methods for treatment of the spine. A preferred embodiment of a system in accordance with the invention is illustrated in connection with FIG. 1.

The intervertebral disc space 10 can be accessed from both sides using two 6.9 mm cannulas 12, 14, for example, which are inserted using standard dilation procedures. Note that other size cannulas can also be used, however, preferred embodiments of the invention utilize one or more cannulas under 80 mm in diameter to minimize trauma to the patient. A tool system 20 is used to insert a plurality of tool instruments into the disc space 10 to remove tissue. Tool system 20 can be connected to computer 65 and can include an imaging device and suction elements. The tool system has a plurality of tools 26 long enough to extend through cannulas into the disc space 10. The user grasps the handle 24 of each tool used to operate switches or manually actuates 22 which operate the directional movement of the articulating tip 21 and/or the tool element 25. The tool can include a cable and/or tubing 28 that can include electrical connection wiring, a suction tube, or fluid delivery and removal tubing to the system 20 which can also house a pump. Following removal of the disc, a balloon delivery system 40 is used to insert inflatable membranes or balloons into the cavity and a fluid management system 50 is used to deliver and remove fluids to inflate the balloons in a selected sequence. The balloon delivery system 40 can be inserted into one of the cannulas 12, 14 using handle 42 with actuators 45 to control fluid flow through tube 44 from system 50. A visualization system 60 can include an insertable probe that is inserted through the second port and/or an endoscope viewing channel which can be inserted through the same port with the tool and/or balloon delivery systems. Thus, a preferred embodiment provides visualization with the articulating tip 21 in combination with tissue removal. The system 60 can include a computer and/or controller 65 and a display 66. The computer 65 can be attached to an endoscope handle 64 and an endoscope tube 62 that is inserted into cannula 12 and/or 14, as needed to visualize the intervertebral space 10. Note that tissue removal can also be conducted through one or both cannulas 12, 14, either separately or in conjunction with tool delivery.

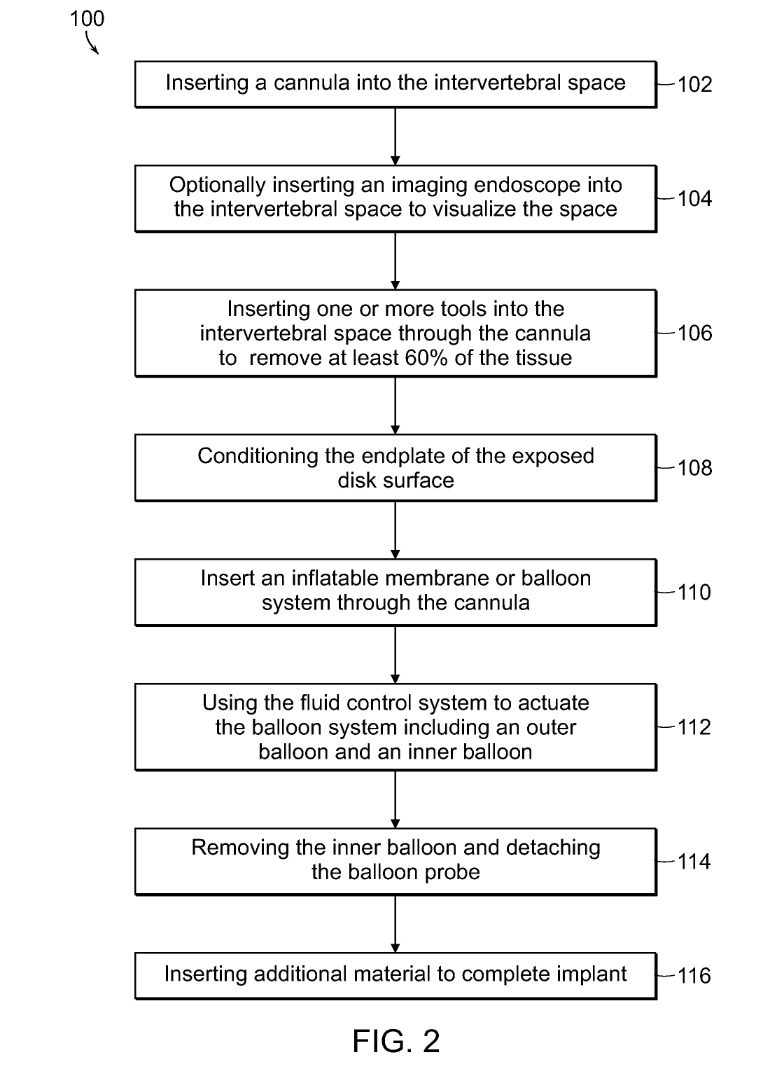

A procedure for performing surgery 100 in accordance with preferred embodiments of the invention is illustrated in FIG. 2. Following dilation of the tissue at a first insertion region, a cannula is inserted 102 into the intervertebral space. Optionally, a second cannula can be inserted at a second insertion region into the intervertebral space. An endoscope can be inserted 104 for visualization of the space during the procedure which the surgeon uses to observe positioning and use of instruments.

A sequence of tools can be used to remove tissue 106. This can involve use of a drill to remove tissue and thereby open a channel to enable the insertion of the end of the cannula into an operative space. The nucleus pulposus is then removed using articulating tools to remove tissue including material from the inner annulus wall surface. The tool system can include suction to remove disc material during the procedure. Alternatively, a fluid delivery and removal system can be included in which a fluid is pumped into and removed from the intervertebral space. This can also improve visualization of the internal space during tissue removal. The resulting annular cavity can have a volume approximately 40 mm×30 mm×12 mm, for example. The precise volume will vary depending on which disc is removed (for example, L1-L2 or L4-L5), whether the individual is male or female, age, etc. Generally the volume is in a range of 10,000 mm³ to 15,000 mm³. Consequently, in adult patients undergoing repair of lumbar discs, it is necessary to remove at least 10,000 mm³ of tissue from the intervertebral space to accommodate the amount of material required for insertion and stabilization of the spine.

An abrading tool, such as a shaver, is used to condition 108 each vertebral endplate such as removal of cartilaginous endplate material to achieve punctate bleeding. Subsequently an implantable device is inserted 110 through the cannula into the annular cavity and a fluid is inserted 112 into the implantable device through the cannula so that the implant conforms to the vertebral space. A preferred embodiment utilizes a probe having a first balloon and a second balloon. The second or inner balloon is temporarily inflated to provide proper spacing between the vertebral bodies. The second balloon is then filled with a second fluid material for permanent placement. The inner balloon is then either filled with cement or collapsed and removed 114. Additional graft or bone cement material can be inserted 116 to complete the implant and seal the annulus.

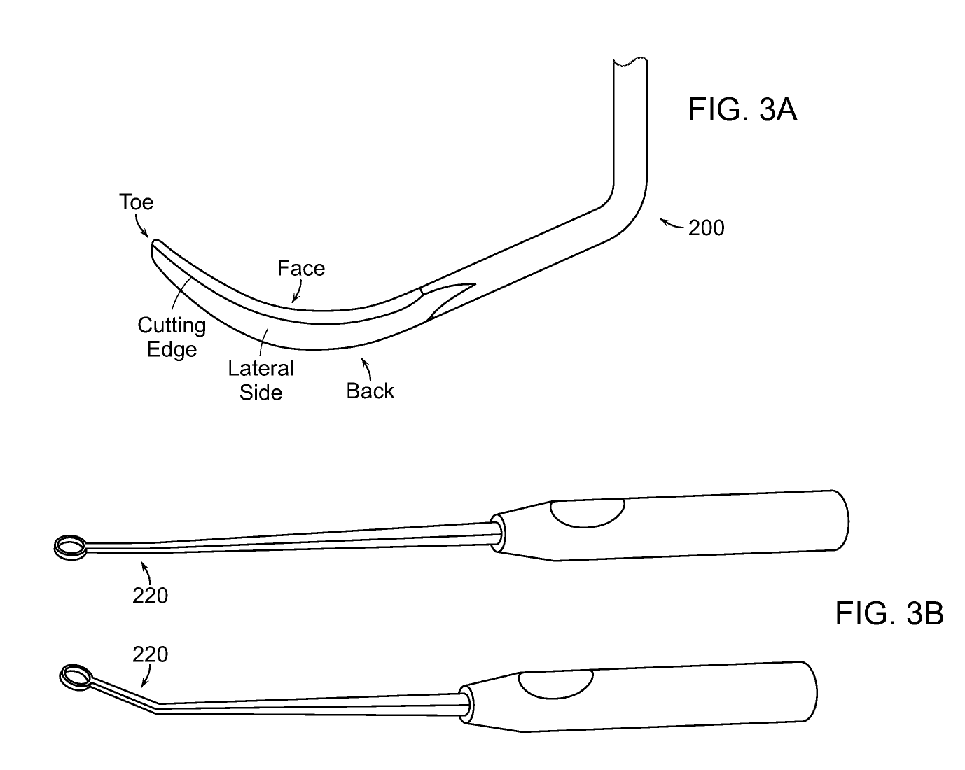

Illustrated in FIGS. 3A-3B are tools, such as a rongeur, curette 200, rake or ring curette, that is inserted through the cannula to remove tissue thereby forming a cavity within the disc space. The curettes 200 can have a rounded back and cutting edges adjacent the face. The ring curette 220 has a distally mounted ring or cup with a cutting edge. Forceps can also be used to grasp and remove disc material. These tool elements can have rigid shafts that extend through the cannula. In order to achieve more complete discectomy, however, the distal end of at least one of the tool elements must be able to bend to a different angle. As it is desirable to minimize damage to the annulus arising from insertion of the cannula, the distal end of the tool can be adjustable, so that the cutting or abrading portion of the tool can be inserted at different lengths and angles to reach different regions of the intervertebral space.

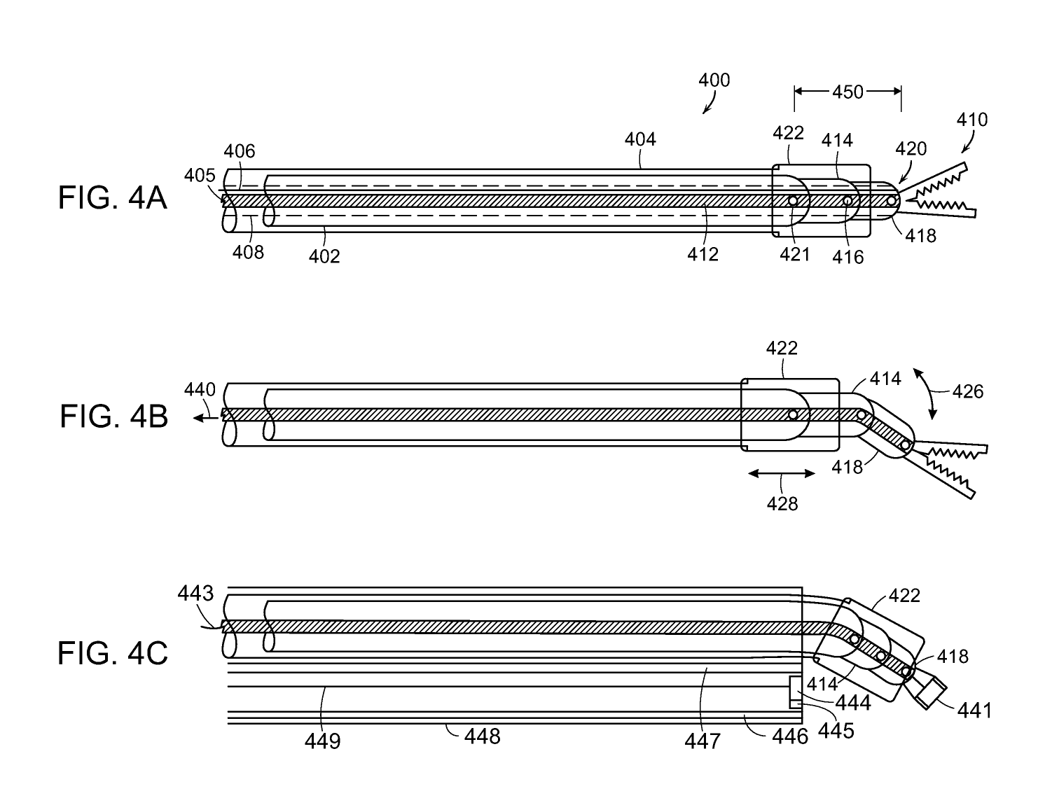

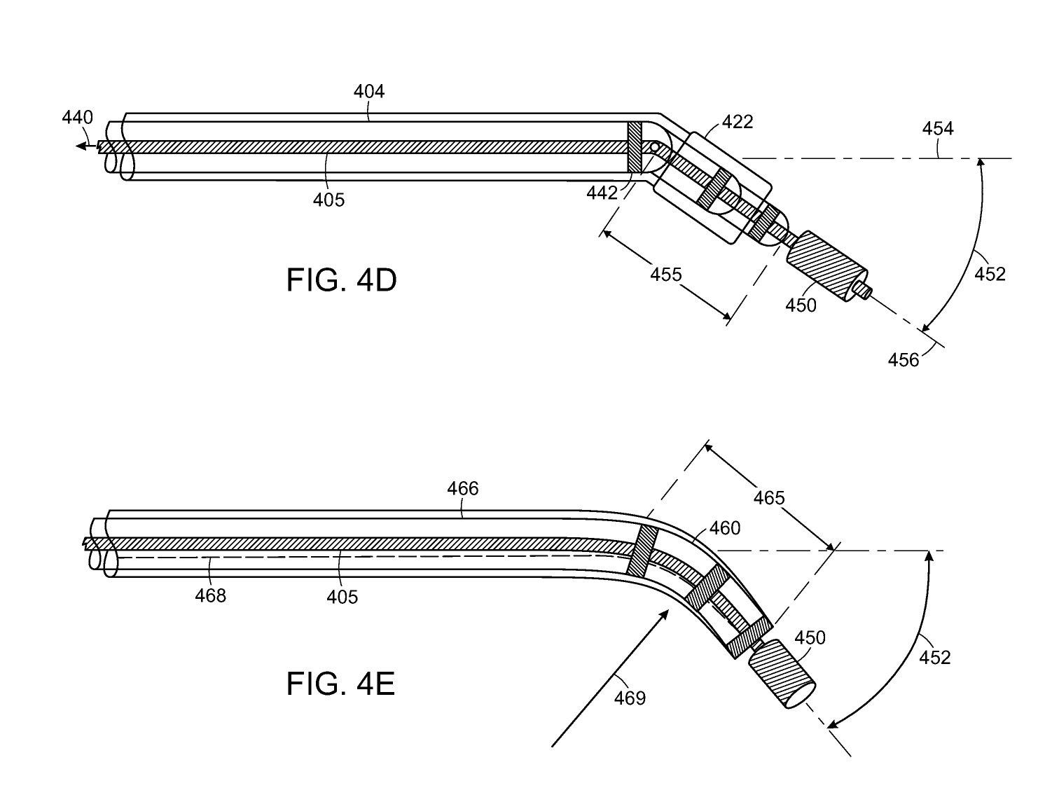

Illustrated in FIGS. 4A-4E is an articulating joint on the distal end of a tool in accordance with preferred embodiments of the invention. This allows the user to adjust the angle of the distal end of the tool relative to the tool axis extending through the cannula. The tool 400 has a first tube 402 attached at first joint 421 to a first rotating member 414. First member 414 is attached at second joint 416 to a second rotating member 418. Although two moving elements 414, 418 are shown, one, two or more elements can be mounted at the distal end of the tube 402, depending on the size of the arcuate movement 426 desired for a particular procedure. The actuators 22 on handle 24 can operate cables 408 to alter the direction of the articulating tip 21. The tip has an articulating or rotating length 450 that is in the range of 10-30 mm such that the tip is capable of extending across the annular cavity within the intervertebral space and have a radius of curvature 460 that is small enough to enable the tool element to rotate within the small annular space and supply a sufficient force to abrade the inner surface.

A preferred embodiment utilizes a release and locking mechanism 422 actuated by the user from the proximal end of the tube 402. When the release mechanism 422 is pulled 428 toward the proximal end of the tube 402, this allows first member 418 to rotate 426 (FIG. 4B). The member can be spring loaded to move and lock into the rotated position or rotated by the user with a cable 408. An outer tube 404 can be used to displace mechanism 422.

A grasping tool 410, such as a rangeur, can be mounted on the distal end with a connector 420, so that different tool elements 25 can be mounted on the distal end. In this example, a control wire 406 extending through the tube 402 is used to actuate the grasping element, such as forceps, or other device mounted at the distal end. A control tube or rod 405 can be used to rotate the tool 410. The tube 405 can incorporate a channel for suction 440. Spacer 442 can position the tube 405 and the central wires

As shown in FIG. 4C, a preferred embodiment can utilize the mechanism 422 to lock the rotating elements 414, 418 into a fixed position. The user can move the mechanism 422 with a tube 404 or other control elements. For applications in which it is desirable to visualize the procedure through the same port or cannula in which the tool system is used, the tool can be introduced through an endoscope channel or can be integrated in a single endoscope body in which different tool elements can be mounted on the distal end. As shown in FIG. 4C these embodiments can utilize an outer tube body 448 in which a distally mounted detector assembly 444 can be mounted at the distal end in which image data is transferred over cable 449 to handle 24. Alternatively, a fiber optic channel can also be used for both delivering illumination from a light source and for light collection and coupling to an external detector. A distal light source 445, such as one or more LEDs, can also be mounted on the distal end. Fluid delivery and removal tubes 447, 446 can be used to flush fluid through the surgical site to remove debris. In this embodiment, an RF electrode 441 is mounted at the distal end to perform electrosurgical procedures. Cable 443 provides the current from handle 24 for this application.

Shown in FIG. 4D is the use of the locking element 422 to position the rotating abrading tool 450 at an angle 452 between the longitudinal axis 454 of the tool and the tool element axis 456. The distal rotation angle 452 can include the angle of just the tool element 450. The angle of the tool and the distal end of the outer tube 404, 460 of the tool 40, or just the angle of the distal end tool tubing. In order to achieve the necessary rotation of the tool within the small cavity being created, the distal end of the tool must be able to bend at an angle of at least 30 degrees.

More generally, the maximum tip angle 452 can be in a range of 30-40 degrees depending upon the location in the cavity. The bending or rotating length 455, that is the length of the distal end of the tool that rotates relative to the tool axis, must be adjustable to reach the desired amount of intervertabral material to be removed. Thus, both the bending length and the bending angle of the distal end of the tool must have a plurality of selectable values to access the different portions of the cavity. Thus, the tip angle can range from zero to at least 25 degrees, and preferably up to a maximum angle in the range of 30-40 degrees.

Shown in FIG. 4E is a preferred embodiment of the invention in which a distal section comprises a compliant or flexible tube 460. The distal section 465 comprises of the flexible tube 460 that can house cables 468 that are used to select an angular position for the tool element 450. The angle of the distal end can be displaced over a selected length 465 and a selected angle 452 to provide a desired radius of curvature 469. The cable system 468 can be actuated by actuators 22 on handle 24 of the tool system. The bending length and bending angle can both have a plurality of selectable values to enable delivery of the selected tool to the plurality of locations needed to remove the required material.

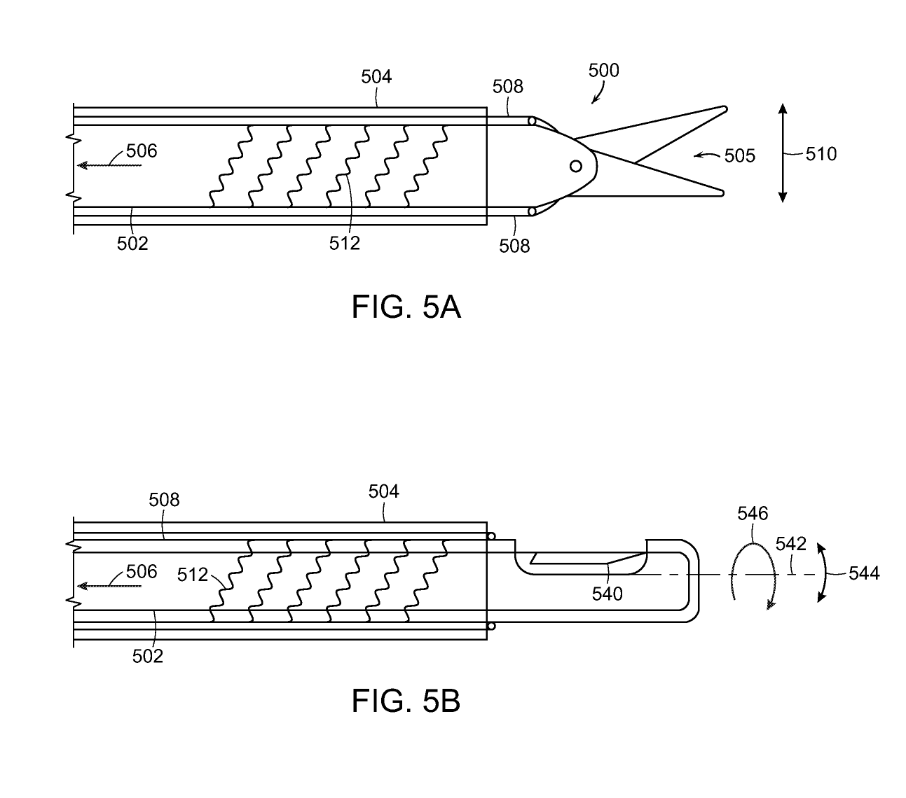

FIG. 5A illustrates a metal tube that both bends and rotates to transmit torque along its length for cutting purposes. The tool 500 can be a cutting scissor 505 mounted on a metal tube 502 with a plastic tube or sheath 504 enclosing the tube 502 so that suction 506 can be applied from the proximal end to remove tissue. Note that a fiber optic probe or endoscope can be inserted within or adjacent to the tube 504 to provide visualization through the same cannula. The tool can also be inserted through an endoscope channel. Steering cables can be used to steer the distal end of steering tube 508 which can rotate 510 through an arc of at least 90 degrees. The inner tube 502 can have a spiral slit structure 512 that enables rotational and bending movement. The tube 502 can include a flexible section made by laser cut interlocking ridges to form structure 512. Tube 502 is positioned in steering tube 508.

In the embodiment of FIG. 5B, a motorized cutting tool such as a burr 540, blade or shaver rotates 546 at adjustable speeds. The longitudinal axis 544 of the tool can be adjusted along an arc 544 as described in detail herein. Suction 506 is used to remove disc material removed by the tool.

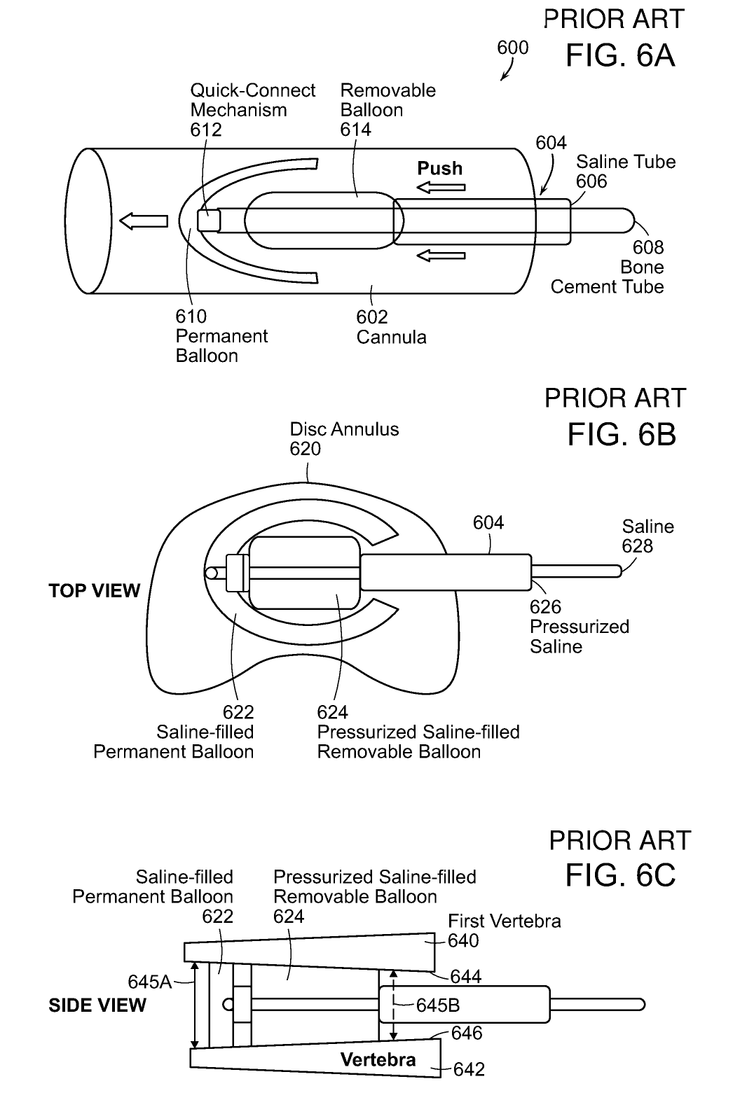

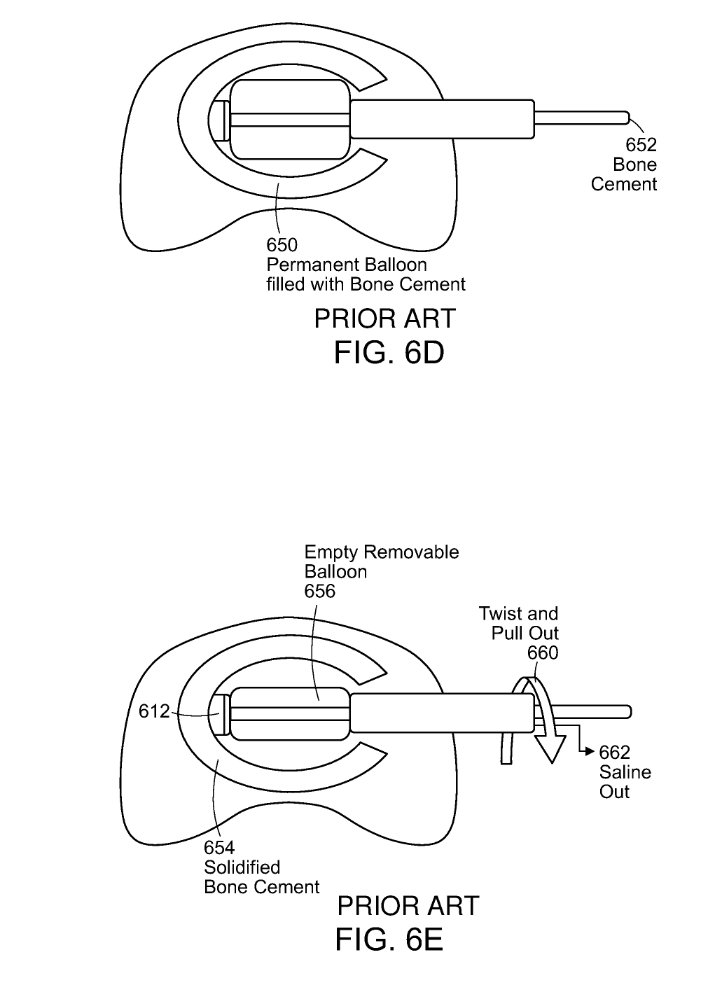

Schematically shown in FIGS. 6A-6E is the process of inserting a balloon system into the intervertebral cavity. A probe 604 is inserted through cannula 602 with a first balloon or membrane 610 mounted on the distal end with a connector 612 that can be quickly released by the user. The balloon can also be released by pulling, cutting or heating. The first balloon 610 is advanced through the cannula 602 in a reduced diameter state for positioning within the disc annulus 620 as shown in FIG. 6B. The probe also has a second balloon 614 mounted proximal to the connector 612. The second balloon 614 is advanced into the cannula 602 in a reduced diameter state. The probe 604 has a first fluid or inner tube 608 that enables the user to direct a fluid through the tube 608 to expand the first balloon to an expanded state 622 that encircles the second balloon 624. A second fluid delivery tube 506 is used to deliver a fluid into the second balloon. Note that the second balloon can be inflated first to separate the vertebra by a selected distance.

In a preferred sequence, first and second balloons are filled with a fluid such as saline to separate first vertebra 640 from second vertebra 642 as shown in the side view of FIG. 6C. The spacing between the associated vertebral endplates 644 and 646 is defined by the amount of fluid that is inserted. Fluoroscopy can be used to verify proper positioning of the balloons to achieve the desired endplate separation. Note that the planes defined by the endplate surfaces are not parallel.

The shape of the balloon is selected to conform to the volume defined by the endplates and the surrounding radius. As shown in FIG. 6C, while the second balloon remains inflated to maintain proper spacing of the endplates, the fluid is removed from the first balloon by suction and a second fluid such as a bone cement 652 is inserted into the first balloon as shown in FIG. 6D to inflate the balloon in its final implant configuration 650. The saline is then removed 662 to empty the second balloon 656 as shown in FIG. 6E. The probe can be twisted 660 to release the connector 612 from the first balloon 654 after the cement has solidified. Alternatively, the balloon can also be released by cutting or pulling the probe or melting a coupling between the probe and the balloon.

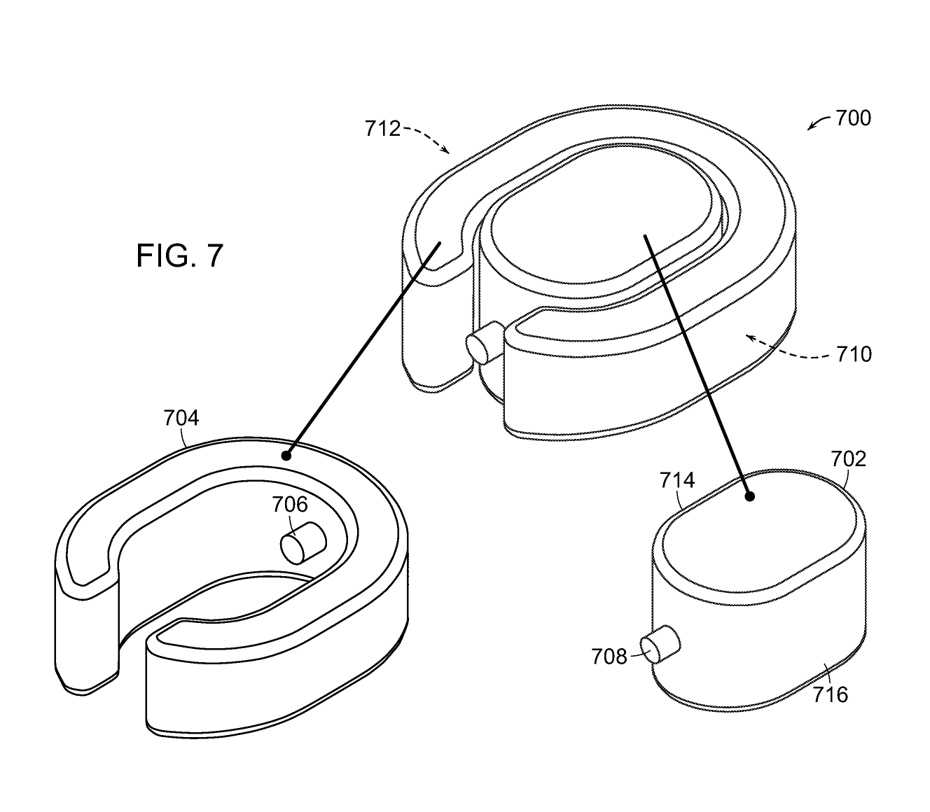

A perspective view of a two balloon system 700 in accordance with the invention is shown in FIG. 7. The outer balloon 704 serves as the implant that must have a reduced diameter of 6.9 mm or less to be inserted through the cannula tube. The reduced diameter can be achieved by folding the balloon to achieve the smaller form factor. The outer balloon must define a contact area of about 110 mm². The contact pressure is generally less than 4.6 MPa to avoid damaging the adjacent vertebra. The first balloon can preferably have a load carrying capacity of 500 Newton to prevent failure under the compressive loads. Preferably, the balloon can exert a propping force of at least 600 Newton to maintain the original disc space with about 2 MPa of pressure. A preferred embodiment of the balloons can be molded using a polyethylene terephthalate (PET) material which has the required tensile strength, stiffness, operating temperature and thickness required for the biocompatible implant. Balloons having the illustrated shapes and sizes can be manufactured using molding techniques. Manufacturing of medical grade balloons and implant materials such as Vention Medical of Salem, N.H. can provide balloons with a variety of shapes and characteristics. The balloon can have a size and shape selected to conform to the vertebral spacing for a particular patient.

The inner, or second balloon, 702 is attached to the probe extending through coupling 708 to the connector 706. Due to the varying distance between the endplate of the adjoining vertebra, a first side 712 can be higher than the second side 710. The second balloon 702 can also have a first sidewall 714 that is higher than the second, or opposite, sidewall 716. The pressure at which each of the respective balloons is inflated can be adjusted separately to maintain proper spacing of the vertebrae. Thus, while the first balloon is deflated the pressure in the second balloon can be increased to maintain proper spacing. When the first balloon is filled with cement, the pressure in the second balloon can be lowered to maintain proper spacing.

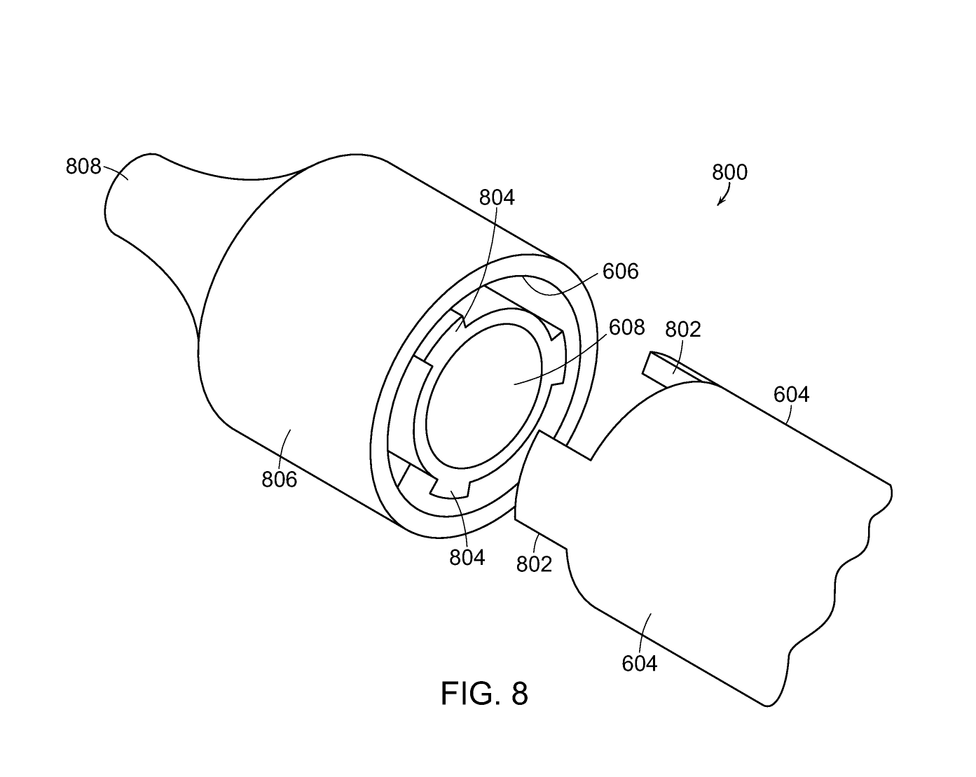

A preferred embodiment of the connector assembly 800 is shown in the perspective view of FIG. 8. The connector port 806 can be permanently attached to the first balloon. The port 806 can utilize a one-way valve 808 at the distal end which has a narrow enough channel to prevent backflow of the more viscous bone cement, while allowing the introduction and removal of saline. The port 806 can utilize connecting elements or tabs 804 that can engage connecting arms 802 on the distal end of probe 604. The bone cement can be inserted under pressure through central lumen 608, while saline can be introduced through peripheral lumen 606, both of which mate to the lumens within tube 604. The user can rotate probe 604 to disengage the arms or tabs 802 from tube 804 to remove the probe from the intervertebral space.

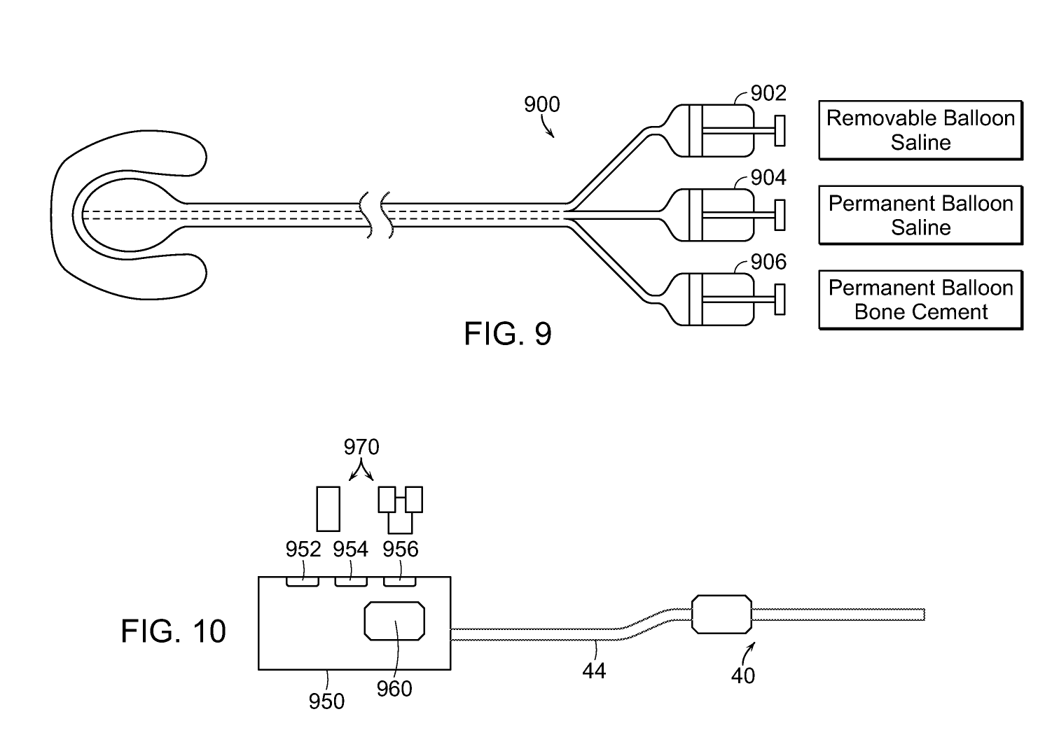

The fluid management system 50 of FIG. 1 can include a combination of syringes used to deliver precise amounts of fluid into the lumens of the balloon delivery system 40. System 40 can include a handle 42 attached to the probe tube 604. The handle 42 can have one or more connectors that couple the lumens within the tube 604 to external tubing 44. As shown in FIG. 9 a plurality of syringes 902, 904, 906 can be used to manually inject the fluid into the handle 42.

Another preferred embodiment of the fluid management system illustrated in FIG. 10 utilizes an automated fluid delivery system 950 that uses separate pumps 952, 954, 956 to deliver precise volumes of fluid at regulated pressures. This system 950 can be monitored and controlled at a control panel 960 or can be connected to a personal computer 60 with display 66 that can display the delivered volume pressure and flow rate of each fluid. The system can also provide for controlled removal and storage of waste material. The fluids can be inserted using disposable cartridges 970. The display 66 can also display images provided by endoscope or fiber optic probe 62 so that the surgeon can view the procedure and injection process on the same monitor. This system enables controlled delivery of fluids into the balloon system to achieve the desired vertebral separation prior to implanting of the material to be solidified within the cavity. The vertebral spacing can typically be different at anterior and posterior sides of the cavity. Thus, the distance 645A can be larger at a first side of the cavity, as shown in FIG. 6C, than the distance 645B on the opposite side of the cavity. The pressure in the two balloons 622, 624 can be modulated relative to each other to maintain the proper distance between the vertebrae. The system can incorporate relief valves into each pressurized fluid delivery tube to prevent any undesired overpressure in the system.

While this invention has been particularly shown and described with references to preferred embodiments thereof, it will be understood by those skilled in the art that various changes in form and detail may be made therein without departing from the spirit and scope of the invention or equivalents thereof as defined by the appended claims.

1. A surgical method for treating a spine comprising:
inserting a first tube into an intervertebral space of a spine;
inserting a tool through the first tube or a second tube and into the intervertebral space;
endoscopically viewing the intervertebral space;
removing tissue from the intervertebral space to provide a cavity;
inserting a first expandable membrane and a second expandable membrane into the cavity with a probe;
inflating the first expandable membrane with a fluid to a selected pressure with a fluid delivery system, the fluid delivery system being configured to control volume and pressure of fluids delivered through the probe;
inflating the second expandable membrane within the cavity with the fluid delivery system;
removing the fluid from the first expandable membrane;
inserting a solidifying fluid into the first expandable membrane with the fluid delivery system to solidify and thereby form a solid implant that separates a first vertebra from a second vertebra; and
removing the second expandable membrane from the cavity.

2. The method of claim 1 further comprising inserting an endoscope into the intervertebral space.

3. The method of claim 1 further comprising actuating an articulating end of a cutting tool to adjust a cutting element at a distal end of the cutting tool.

4. The method of claim 1 wherein the endoscopically viewing step further comprises inserting a device for imaging into the intervertebral space through the second tube.

5. The method of claim 1 further comprising removing at least 60 percent of tissue within the intervertebral space.

6. The method of claim 1 wherein the first tube has an outer diameter of less than 8 mm.

7. The method of claim 1 further comprising inserting a prosthetic material into the intervertebral space around the solid implant.

8. The method of claim 1 further comprising suctioning tissue from the intervertebral space.

9. The method of claim 1 further comprising inflating the first expandable membrane including a first balloon to obtain a selected spacing between a first vertebra and a second vertebra.

10. The method of claim 1 further comprising removing at least 80 percent of tissue within the intervertebral space.

11. The method of claim 1 further comprising using an irrigation fluid to remove tissue from the cavity.

12. The method of claim 2 further comprising removing at least 60 percent of the tissue in the intervertebral space by selectively adjusting a bending length and a bending angle of an articulating end on a tissue removal tool positioned at a distal end of the first tube or the second tube.

13. The method of claim 1 wherein the probe comprises a membrane delivery system wherein the second expandable membrane is positioned such that the first expandable membrane extends around at least a portion of the second expandable membrane.

14. The method of claim 1 further comprising coupling a membrane inflation tube device within the probe to the first expandable membrane and the second expandable membrane.

15. The method of claim 1 further comprising visualizing the cavity with an endoscope inserted with the second tube that comprises a second cannula and a tissue removal device that is inserted through the first tube that comprises a first cannula.

16. The method of claim 1 further comprising removing tissue with a cutting device selected from the group comprising an RF electrode, a moving blade, or a manually operated tool.

17. The method of claim 1 further comprising inflating the first expandable membrane or the second expandable membrane with the fluid delivery system coupled to the probe the first expandable membrane remaining in the intervertebral space after surgery.

18. The method of claim 1 further comprising removing tissue from the intervertebral space with an endoscopic tissue removal system.

19. The method of claim 18 wherein the fluid delivery system comprises a housing containing a plurality of fluids, each fluid being delivered to the first expandable membrane or the second expandable membrane.

20. The method of claim 18 wherein the fluid delivery system comprises a first fluid source, a second fluid source and a control system.

21. The method of claim 1 wherein the first tube is inserted at a first position and wherein an endoscope is inserted through the second tube at a second position and further comprising a tissue removal system having an imaging device positioned at a distal end of an endoscope body of the endoscope wherein a surgical tool can be positioned and visualized at the distal end of the endoscope body.
